(19) United States
(12) Patent Application Publication
(10) Pub. No.: US 2012/0135034 A1
(43) Pub. Date: May 31, 2012

(54) Non-Integrating Retroviral Vector Vaccines

(76) Inventor: Boro Dropulic, Ellicott City, MD (US)

(21) Appl. No.: 13/256,216

(22) PCT Filed: Mar. 13, 2010
(86) PCT No.: PCT/US2010/027262

§ 371 Date: Feb. 20, 2012

Related U.S. Application Data
(60) Provisional application No. 61/160,285, filed on Mar. 13, 2009.
(60) Provisional application No. 61/166,769, filed on Apr. 5, 2009.
(60) Provisional application No. 61/167,088, filed on Apr. 6, 2009.

(51) Int. Cl. A61K 39/295; A61K 39/21; C07K 14/005; A61P 35/00; A61P 37/04; A61P 31/04; A61P 31/12; C12N 15/63; A61K 39/12

(52) U.S. Cl. 424/201.1; 435/320.1; 424/204.1; 530/350; 424/202.1

(57) ABSTRACT

This invention relates to non-integrating, non-replicating retroviral vectors that cause an immune response in an animal host when administered to the host. The vectors transduce cells in the host, where they produce virus-like particles (VLPs), which stimulate an additional immune response in the host when they are released from the cells. The vectors are non-integrating, non-replicating retroviral vectors comprising long terminal repeats, a packaging sequence, and a heterologous promoter operably linked to one or more polynucleotide sequences that together encode the structural proteins of a virus. Methods of making and using the vectors are also disclosed.

### CROSS-REFERENCE TO RELATED PATENT APPLICATIONS

This application claims the benefit of and priority to U.S. Provisional Patent Application No. 61/160,285, filed Mar. 13, 2009, No. 61/166,769, filed Apr. 5, 2009, and No. 61/167,088, filed Apr. 6, 2009, all of which are incorporated herein by reference in their entirety.

### FIELD OF THE INVENTION

This invention relates to vaccines and in particular to non-integrating, replication-incompetent retroviral vectors that induce an immune response in an animal host when administered to the host. The vectors of the invention also transduce cells in the host, where they produce virus-like particles (VLPs), which stimulate an additional immune response in the host.

### BACKGROUND

Retroviral vectors are well-known to persons skilled in the art. They are enveloped virion particles derived from retroviruses that are infectious but non-replicating. They contain one or more expressible polynucleotide sequences. Thus, they are capable of penetrating a target host cell and carrying the expressible sequence(s) into the cell, where they are expressed. Because they are engineered to be non-replicating, the transduced cells do not produce additional vectors or infectious retroviruses.

Retroviruses are enveloped RNA viruses that belong to the family Retrovirida. After infecting a host cell, the RNA is transcribed into DNA via the enzyme reverse transcriptase. The DNA is then incorporated into the cell's genome by an integrase enzyme and thereafter replicates as part of the host cell's DNA. The Retrovirida family includes the genera Alpharetrovirus, Betaretrovirus, Gammaretrovirus, Deltaretrovirus, Epsilonretrovirus, Lentivirus, and Spumavirus.

Retroviral vectors derived from Gammaretroviruses are well known to the art and have been used for many years to deliver genes to cells. Such vectors include ones constructed from murine leukemia viruses, such as Moloney murine leukemia virus, or feline leukemia viruses.

Lentiviral vectors derived from Lentiviruses are also well known to the art. They have an advantage over retroviral vectors in being able to integrate their genome into the genome of non-dividing cells. Lentiviruses include human immunodeficiency virus (HIV), simian immunodeficiency virus (SIV), bovine immunodeficiency virus, equine infectious anemia virus, feline immunodeficiency virus, puma lentivirus, caprine arthritis encephalitis virus, and visna/maedi virus.

These vectors, being foreign antigens, produce an immune response in an animal host. The present invention uses this response to create a desirable immunity in a mammal. The non-integrating vectors (NIVs) of the invention are self-boosting vaccines. The retroviral vector particle not only acts as a vaccine itself, but it also produces antigenic VLPs after entering the cells, since it encodes for VLP production from its non-integrating genome. This provides a second round of immune stimulation.

VLPs are not viruses. They consist only of an outer viral shell and do not have any viral genetic material. Thus, they do not replicate. The expression of capsid proteins of many viruses leads to their spontaneous assembly into supramolecular, highly repetitive, icosohedral or rod-like particles similar to the native virus they are derived from but free of viral genetic material. Thus, VLPs represent a non-replicating, non-infectious particle antigen delivery system that stimulates both native and adaptive immune responses. Being particulate, they provide the critical “danger signal” that is important for the generation of a potent and durable (after multiple immunizations) immune response. VLPs can be extremely diverse in terms of the structure, consisting of single or multiple capsid proteins either with or without lipid envelopes. The simplest VLPs are non-enveloped and assemble by expression of just one major capsid protein, as shown for VLPs derived from hepadnaviruses, papillomaviruses, parvoviruses, or polyomaviruses.

NIVs are similar to VLPs, except that they also contain genetic information that can express the proteins comprising VLPs, upon entry into cells. Therefore, not only is the NIV itself a VLP vaccine (having a core and antigens comprised within a particle), but upon entry into cells after administration to the host animal, the viral genetic information efficiently enters the nucleus without integration. Here it expresses to high levels proteins that are then assembled to make VLP particles inside the body, amplifying the immunogenic effect. This results not only in a strong primary immune response but a persistent one that can generate long lasting immunity.

A further advantage of NIV vaccines is the small amount needed to generate an immune response. Since the particles are amplified after being produced from cells in the body, the amount of initial material needed to generate an immune response is very small, dramatically improving the economics of such a vaccine.

A vaccine for the prevention of AIDS has been a challenging goal. Protein subunit vaccines have been shown to be ineffective. While live, attenuated HIV's have shown promise in animal studies, their pathogenicity has prevented their further development. Viral vectors have also been used for the development of candidate HIV vaccines. The most notable was the Merck adenoviral vector-based AIDS vaccine that recently failed in human clinical trials. Not only did the vaccine fail to decrease HIV transmission or viral replication in infected subjects, but vaccinated individuals who had been previously exposed to the adenovirus strain used to make the vaccine had an apparent increase in susceptibility to HIV infection. While the reasons for the failure are not known, previous animal studies have demonstrated that anti-vector immunity produced a bifurcation of the immune response, decreasing the potency of the vaccine after multiple injections.

The NIVs of the invention should be especially promising as AIDS vaccines. A non-integrative HIV vector would have all of the attributes of conventional HIV vectors, except that the genome would be maintained as an episome in non-dividing cells for a period of time sufficient to generate VLPs, and would be diluted upon cell division. The HIV NIV particle itself would be a vaccine. Additionally, it would transduce cells, express HIV proteins, and further produce more HIV VLPs to enhance the vaccine's effect. Since the NIV particle will only go through one round of replication and generate only VLPs without any genetic material, they would mimic HIV infection, without the harmful sequelae following live virus exposure.

### SUMMARY OF THE INVENTION

This invention relates to non-integrating, non-replicating retroviral vectors that cause an immune response in an animal host when administered to the host. The vectors transduce cells in the host, where they produce virus-like particles (VLPs), which stimulate an additional immune response in the host when they are released from the cells. The retroviral vectors comprise long terminal repeats, a packaging sequence, and a heterologous promoter operably linked to one or more polynucleotide sequences that together encode the structural proteins of a virus. In one embodiment, the retroviral vectors are lentiviral vectors. The invention also includes pharmaceutical compositions comprising one or more of the vectors of the invention in a pharmaceutically acceptable carrier.

The invention includes plasmids, helper constructs, and producer cells used to construct and produce the vectors. The plasmid comprises retroviral long terminal repeat sequences, a retroviral packaging sequence, and a heterologous promoter operably linked to one or more polynucleotide sequences that together encode the structural proteins of a virus. In one embodiment, the retroviral sequences are lentiviral sequences. In one aspect of this embodiment, the lentiviral sequences are HIV sequences. The packaging cell comprises the plasmid of the invention and a helper construct that does not contain an integrase gene or contains an integrase gene that is not functional. In one embodiment, the cell is a mammalian cell. The producer cell comprises the plasmid of the invention and a helper construct that does not contain an integrase gene or contains an integrase gene that is not functional. In one embodiment, the cell is a mammalian cell.

The vectors and pharmaceutical compositions of the invention are used as vaccines. Thus, the invention includes a method of causing an immune response in a mammal by delivering the vectors or the pharmaceutical compositions to the mammal in an amount sufficient to cause an immune response in the mammal. After the vectors transduce cells in the mammal, the cells produce and release VLPs comprising the structural proteins of the virus, causing a further immune response in the mammal. The VLPs comprise the structural proteins of a target virus. The virus is any virus for which the vectors of the invention can produce self-assembling structural proteins that form a VLP.

### BRIEF DESCRIPTION OF THE FIGURES

FIG. 1 shows examples of some of the NIVs of the invention. LTR: Long Terminal Repeat. Psi: Packaging sequence (leader plus a fragment of the Gag sequence). AS: optional antisense targeted to 300 bp of the highly conserved native integrase gene sequence (or other sequence) to prevent recombination with wt-HIV; RRE: rev responsive element. P: promoter used to drive antigen expression (probably either CMV, SFFV or Tet-O promoter). Gag-Pol: codon optimized Gag and protease gene (avoid codon optimization in the frameshift region), a defective integrase and optionally a defective Reverse Transcriptase, the latter may optionally be deleted from the construct, if desired, from certain versions of the constructs. 2A: the 2A cleavage sequence allows each protein to be cleaved post-translation; use of three 2A in one Lentiviral vector has been demonstrated. Env: the native envelope sequence of SIV that is codon optimized; different envelope strains can be used to broaden immune response. CTL Epitope is a polynuceotide sequence encoding the composite CTL and/or humoral epitoptes from any protein of the target virus. For HIV this could be any combination of the proteins Vpx/Vpr/Vif/Nef*/Tat/Rev/: polypeptide sequence; each polypeptide is present as a codon optimized sequence that is aligned sequentially; no need to express these proteins individually; the Nef*gene sequence has been mutated in the kinase domain; the CTL sequence is a polypeptide sequence that consolidates all the major epitopes into a single polypeptide sequence; whole Vpx is inserted at the N terminus of the polyprotein to facilitate its incorporation into VLP particles. GFP: Green Fluorescent Protein gene used for marking of cell in vitro and in vivo. TMPK: example of a safety gene; thymidylate kinase (TMPK) that phosphorylates 3′-azido-3′-deoxythymidine (AZT) resulting in capase-3 activation and apoptosis (others can be used such as TK, dCK etc. IL-12: Interleukin 12 gene used to promote memory T cell responses; other cytokines, proteins and/or RNAi can be used. SIN-LTR: self-inactivating LTR, which is double-copied during reverse transcription (RT provided as a protein in virion by helper). P-CMV: Cytomegalovirus Promoter. Gag-Pol: the helper plasmid expressing all structural proteins and enzymatic proteins for virion formation and RT, but integrase negative (black bar); the Gag-Pol has been codon degenerated to limit sequence similarity with the Psi sequence in the SIV NIV. Hetero env: eg. VSV-g, Dengue E protein, HA, gp120, Flavivirus Env proteins, any heterologous env capable of pseudotyping with lentiviral vectors.

FIG. 2 shows a NIV Helper Plasmid construct that is used for the production of NIVs that express a core proteins that are not retroviral vector core proteins. They express the Gag proteins of the retrovirus and Pol genes, but express a defective Integrase molecule. The Helper construct is not limited to a particular plasmid, but could have other variations, including other HIV proteins. The helper could also be integrated into a cell line so that the result is a packaging cell line. If the packaging cell line contains a vector, then this would result in a producer cell line.

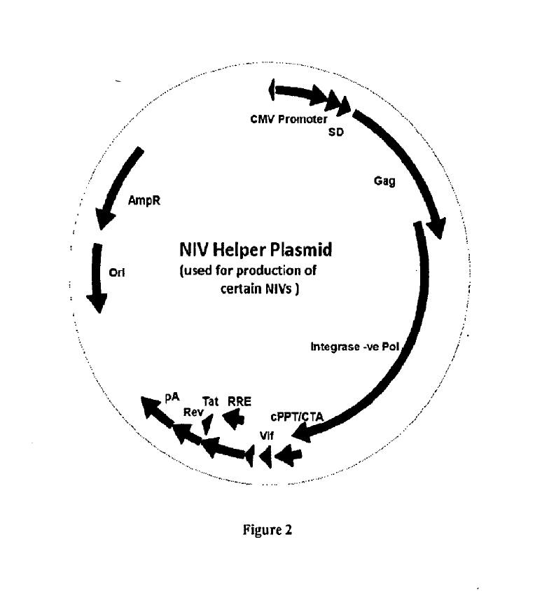

FIG. 3 shows a Dengue Fever Virus NIV vaccine construct that is comprised of a 5′LTR with a psi packaging sequence, RRE element and optionally some non-coding Tat sequence. The Cytomegalovirus promoter (SCMV) drives the expression of the Dengue Core proteins. The Phosphoglucokinase (PGK) promoter drives the expression of the Dengue envelope protein(s) which can be a single ORF or multiple ORFs, separated by 2A or IRES sequences (not shown). The Post Transcriptional Regulatory Element (PRE) is optionally inserted distal to the Envelope ORF and poly A.

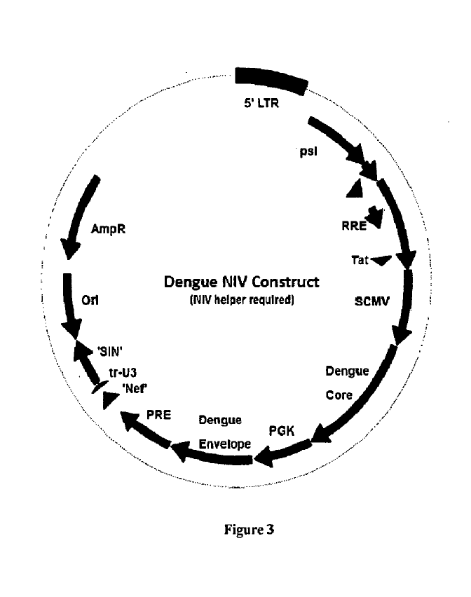

FIG. 4 shows a Hepatitis C Virus NIV vaccine construct that is comprised of a 5′LTR with a psi packaging sequence, RRE element and optionally some non-coding Tat sequence. The Cytomegalovirus promoter (SCMV) drives the expression of the Hepatitis C Core proteins. The Phosphoglucokinase (PGK) promoter drives the expression of the Hepatitis C E1 or E2 envelope protein(s). The Post Transcriptional Regulatory Element (PRE) is optionally inserted distal to the Envelope ORF and poly A.

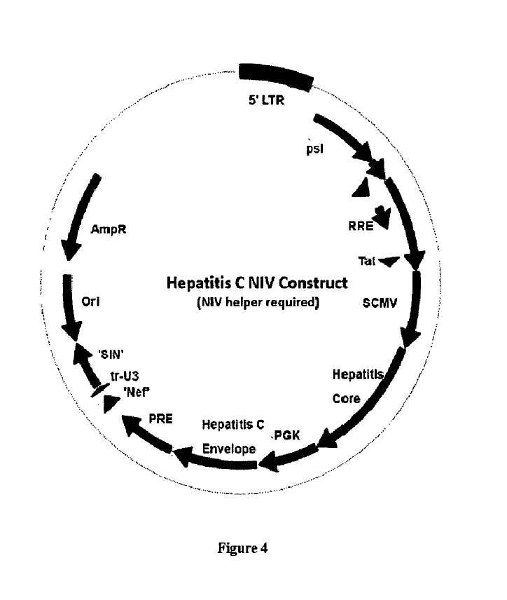

FIG. 5 shows a Hepatitis C Virus NIV vaccine construct that is comprised of a 5′LTR with a psi packaging sequence, RRE element and optionally some non-coding Tat sequence. The Cytomegalovirus promoter (SCMV) drives the expression of the Hepatitis C Core proteins. The Phosphoglucokinase (PGK) promoter drives the expression of the Hepatitis C E1 and E2 envelope protein(s) which are separated by 2A or IRES sequences (not shown). The Post Transcriptional Regulatory Element (PRE) is optionally inserted distal to the Envelope ORF and poly A.

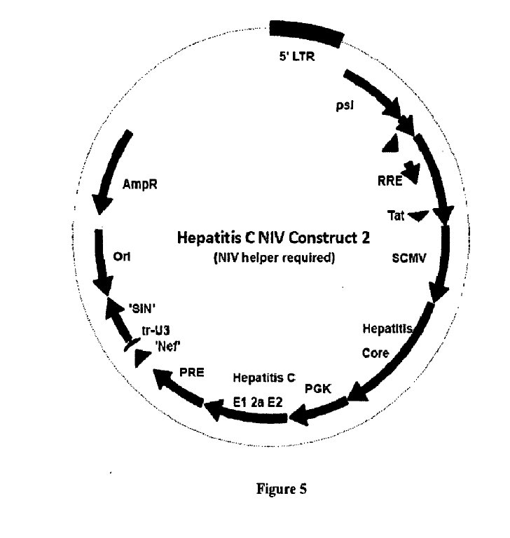

FIG. 6 shows a Hepatitis B Virus NIV vaccine construct that is comprised of a 5′LTR with a psi packaging sequence, RRE element and optionally some non-coding Tat sequence. The Cytomegalovirus promoter (SCMV) drives the expression of the Hepatitis C Core proteins. The Phosphoglucokinase (PGK) promoter drives the expression of the Hepatitis B E1 and/or E2 envelope protein(s) which are separated by 2A or IRES sequences (not shown). The Post Transcriptional Regulatory Element (PRE) is optionally inserted distal to the Envelope ORF and poly A.

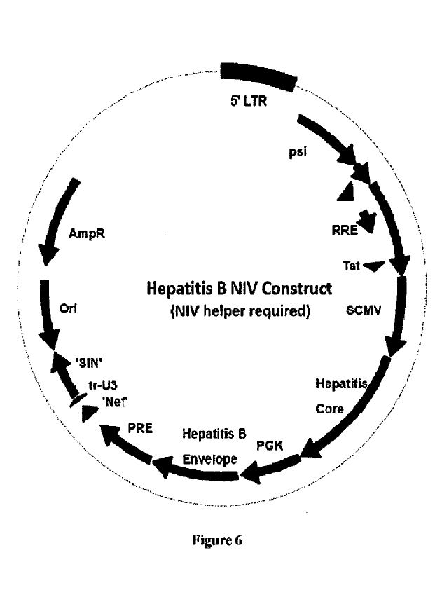

FIG. 7 shows a Influenza Virus NIV vaccine construct that is comprised of a 5′LTR with a psi packaging sequence, RRE element and optionally some non-coding Tat sequence. The Cytomegalovirus promoter (SCMV) drives the expression of the Influenza virus Core (at least M1 but optionally also M2) proteins. The Phosphoglucokinase (PGK) promoter drives the expression of the Influenza C HA and NA envelope protein(s) which are separated by 2A or IRES sequences (not shown). The Post Transcriptional Regulatory Element (PRE) is optionally inserted distal to the Envelope ORF and poly A.

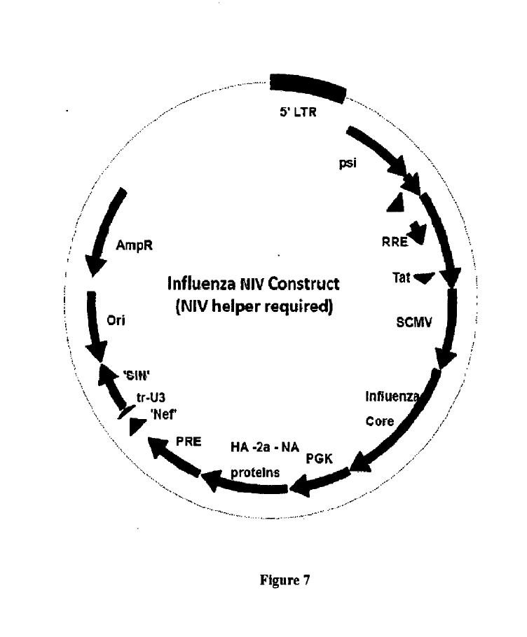

FIG. 8 shows a Influenza Virus NIV vaccine construct that is comprised of a 5′LTR with a psi packaging sequence, RRE element and optionally some non-coding Tat sequence. The Cytomegalovirus promoter (SCMV) drives the expression of the Influenza virus Core (at least M1 but optionally also M2) proteins. The Phosphoglucokinase (PGK) promoter drives the expression of a plurality of Influenza HA envelope protein(s) which differ by strain and are separated by 2A or IRES sequences (not shown). The Post Transcriptional Regulatory Element (PRE) is optionally inserted distal to the Envelope ORF and poly A.

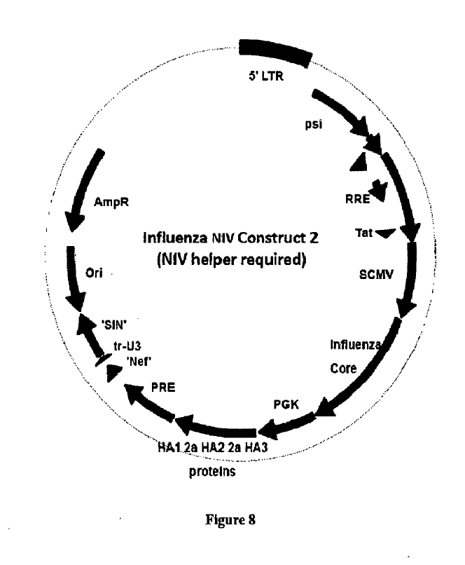

FIG. 9 shows a Tumor Antigen NIV vaccine construct that is comprised of a 5′LTR with a psi packaging sequence, RRE element and optionally some non-coding Tat sequence. The Cytomegalovirus promoter (SCMV) drives the expression of the Influenza virus Core (at least M1 but optionally also M2) proteins. The Phosphoglucokinase (PGK) promoter drives the expression of the Tumor antigen(s), which is preferably a membrane protein(s) which are separated by 2A or IRES sequences (not shown). The Post Transcriptional Regulatory Element (PRE) is optionally inserted distal to the Envelope ORF and poly A.

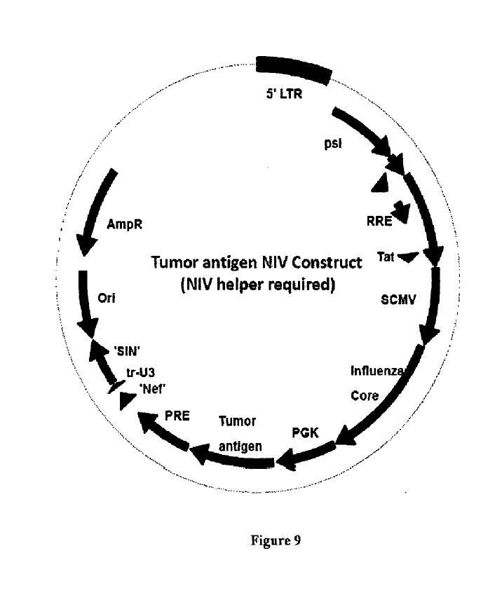

FIG. 10 shows a Bacterial Antigen NIV vaccine construct that is comprised of a 5′LTR with a psi packaging sequence, RRE element and optionally some non-coding Tat sequence. The Cytomegalovirus promoter (SCMV) drives the expression of the Influenza virus Core (at least M1 but optionally also M2) proteins. The Phosphoglucokinase (PGK) promoter drives the expression of the bacterial antigen(s) which are anchored into the envelope by fusion to the HA transmembrane domain to make a chimeric protein. Each antigen is separated by 2A or IRES sequences (not shown). The Post Transcriptional Regulatory Element (PRE) is optionally inserted distal to the Envelope ORF and poly A.

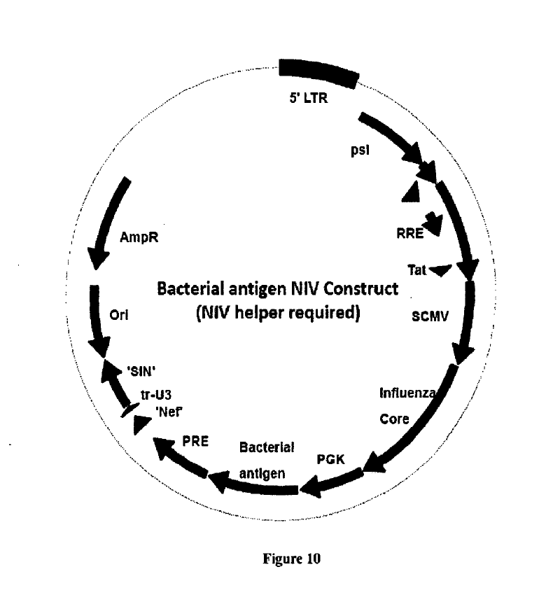

FIG. 11 shows a Tumor Antigen NIV vaccine construct 2 that is comprised of a 5′LTR with a psi packaging sequence, RRE element and optionally some non-coding Tat sequence. The Cytomegalovirus promoter (SCMV) drives the expression of the Influenza virus Core (at least M1 but optionally also M2) proteins. The Phosphoglucokinase (PGK) promoter drives the expression of the Tumor antigen and a cytokine, which are separated by 2A or IRES sequences (not shown). The Post Transcriptional Regulatory Element (PRE) is optionally inserted distal to the Envelope ORF and poly A.

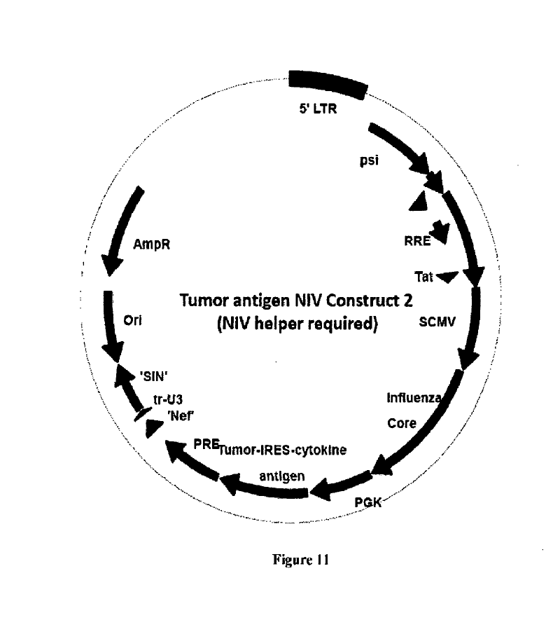

FIG. 12 shows a HIV NIV vaccine construct that is comprised of a 5′LTR with a psi packaging sequence, RRE element and optionally some non-coding Tat sequence. The Cytomegalovirus promoter (SCMV) drives the expression of the HIV Gag and Protease Core proteins. The Phosphoglucokinase (PGK) promoter drives the expression of the HIV envelope protein and a CTL epitope polypeptide (encoding for major CTL epitopes on the HIV genome) which are separated by 2A or IRES sequences (not shown). The Post Transcriptional Regulatory Element (PRE) is optionally inserted distal to the Envelope ORF and poly A.

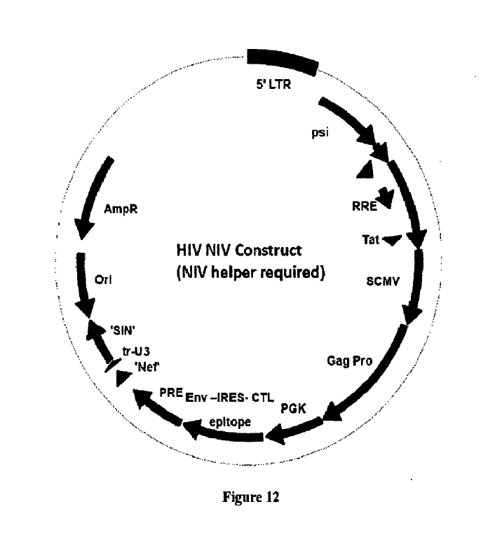

FIG. 13 shows a HIV/AIDS NIV vaccine construct that is comprised of a 5′LTR with a psi packaging sequence, RRE element and optionally some non-coding Tat sequence. The Cytomegalovirus promoter (SCMV) drives the expression of the HIV Gag and Pol Core proteins, where the Integrase is mutated and not functional. The Phosphoglucokinase (PGK) promoter drives the expression of the HIV envelope protein. The Post Transcriptional Regulatory Element (PRE) is optionally inserted distal to the Envelope ORF and poly A.

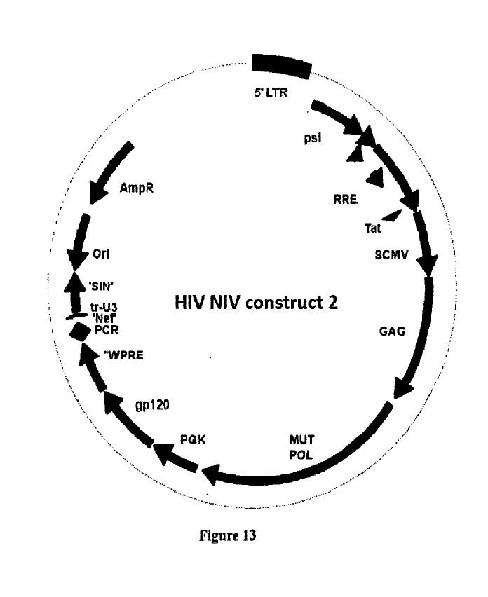

FIG. 14 shows a HIV/AIDS NIV vaccine construct that is comprised of a 5′LTR with a psi packaging sequence, RRE element and optionally some non-coding Tat sequence. The Cytomegalovirus promoter (SCMV) drives the expression of the HIV Gag and Pol Core proteins, where the Integrase is mutated and not functional. The Phosphoglucokinase (PGK) promoter drives the expression of the HIV envelope protein and a CTL epitope polypeptide (encoding for major CTL epitopes on the HIV genome) which are separated by 2A or IRES sequences (not shown). The Post Transcriptional Regulatory Element (PRE) is optionally inserted distal to the Envelope ORF and poly A.

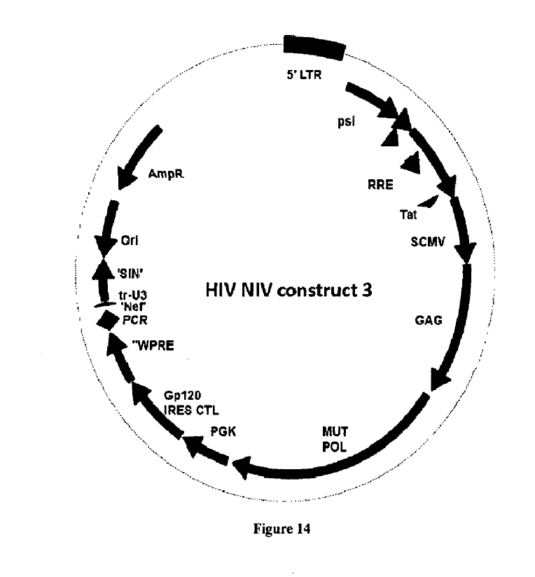

FIG. 15 shows a HIV/AIDS NIV vaccine construct that is comprised of a 5′LTR with a psi packaging sequence, RRE element and optionally some non-coding Tat sequence. The Cytomegalovirus promoter (SCMV) drives the expression of the HIV Gag and Pol Core proteins, where the Integrase is mutated and not functional. The Phosphoglucokinase (PGK) promoter drives the expression of the HIV envelope protein and a cytokine (eg IL12) which are separated by 2A or IRES sequences (not shown). The Post Transcriptional Regulatory Element (PRE) is optionally inserted distal to the Envelope ORF and poly A.

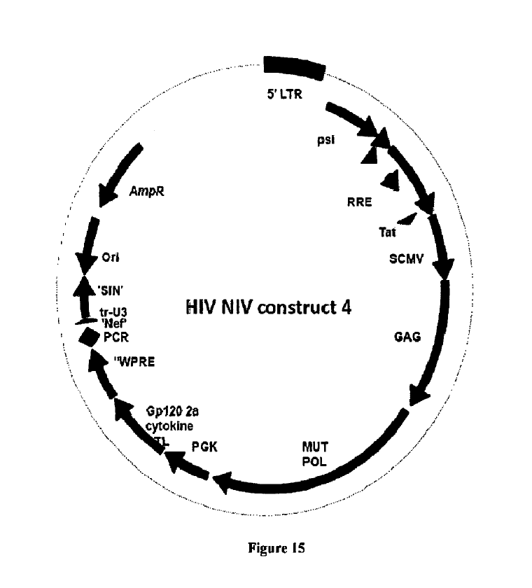

FIG. 16 shows a HIV/AIDS NIV vaccine construct that is comprised of a 5′LTR with a psi packaging sequence, RRE element and optionally some non-coding Tat sequence. The Cytomegalovirus promoter (SCMV) drives the expression of the HIV Gag and Pol Core proteins, where the Integrase is mutated and not functional. The Phosphoglucokinase (PGK) promoter drives the expression of the HIV envelope protein, a CTL epitope polypeptide (encoding for major CTL epitopes on the HIV genome), a cytokine (eg GCSF) and a cell death inducing gene (eg. TMPK), which are all separated by 2A or IRES sequences (not shown). The Post Transcriptional Regulatory Element (PRE) is optionally inserted distal to the Envelope ORF and poly A.

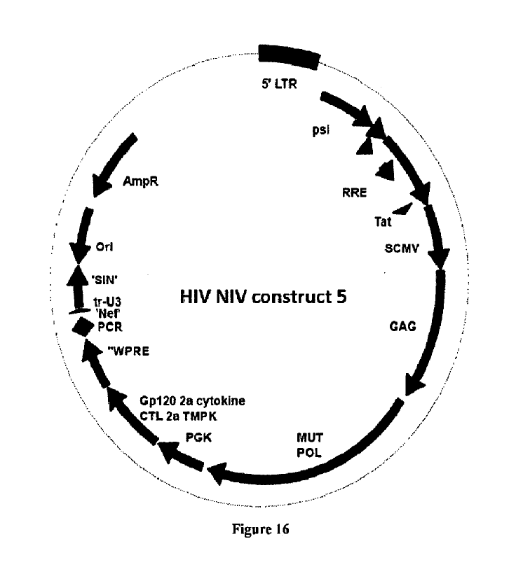

FIG. 17 shows a Hepatitis C NIV vaccine construct that is comprised of a 5′LTR with a psi packaging sequence, RRE element and optionally some non-coding Tat sequence. The Cytomegalovirus promoter (SCMV) drives the expression of the HIV Gag and Pol Core proteins, where the Integrase is mutated and not functional. The Phosphoglucokinase (PGK) promoter drives the expression of the Hepatitis C E1 and E2 envelope proteins, which are all separated by 2A or IRES sequences (not shown). The Post Transcriptional Regulatory Element (PRE) is optionally inserted distal to the Envelope ORF and poly A.

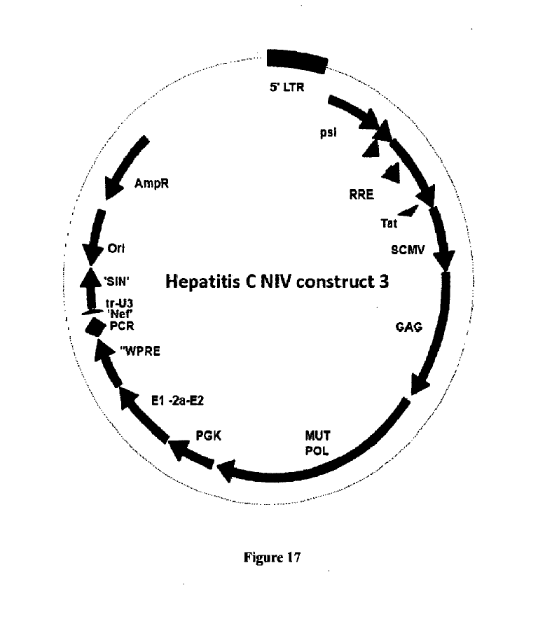

FIG. 18 shows a Tumor Antigen NIV vaccine construct that is comprised of a 5′LTR with a psi packaging sequence, RRE element and optionally some non-coding Tat sequence. The Cytomegalovirus promoter (SCMV) drives the expression of the HIV Gag and Pol Core proteins, where the Integrase is mutated and not functional. The Phosphoglucokinase (PGK) promoter drives the expression of the Tumor antigen proteins, which if plural, are all separated by 2A or IRES sequences (not shown). The Post Transcriptional Regulatory Element (PRE) is optionally inserted distal to the Envelope ORF and poly A.

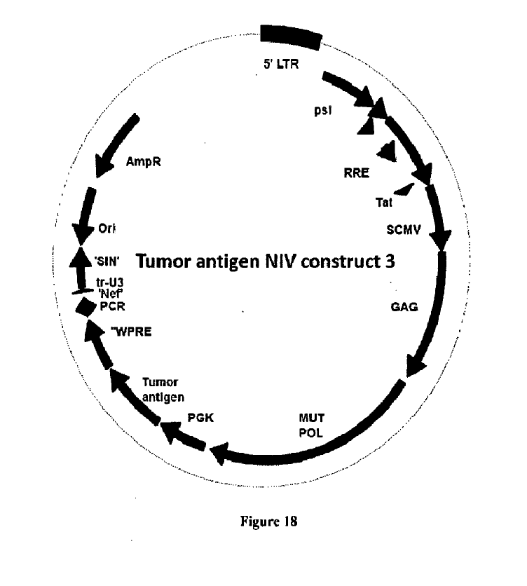

FIG. 19 shows a Dengue Fever NIV vaccine construct that is comprised of a 5′LTR with a psi packaging sequence, RRE element and optionally some non-coding Tat sequence. The Cytomegalovirus promoter (SCMV) drives the expression of the HIV Gag and Pol Core proteins, where the Integrase is mutated and not functional. The Phosphoglucokinase (PGK) promoter drives the expression of Dengue E proteins, which if plural, are all separated by 2A or IRES sequences (not shown). The Post Transcriptional Regulatory Element (PRE) is optionally inserted distal to the Envelope ORF and poly A.

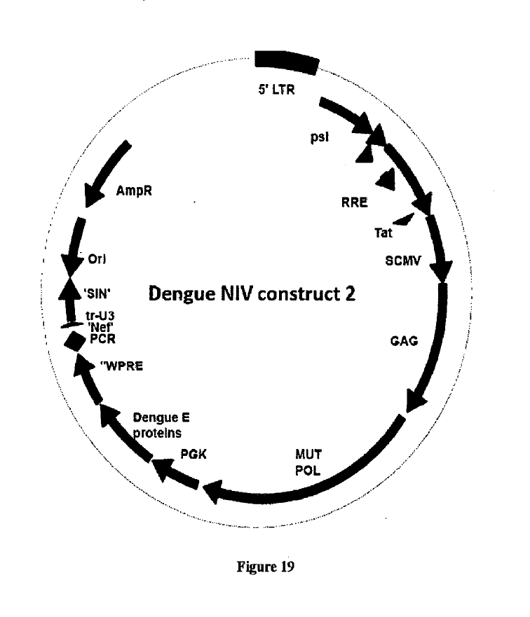

FIG. 20 shows a Malaria NIV vaccine construct that is comprised of a 5′LTR with a psi packaging sequence, RRE element and optionally some non-coding Tat sequence. The Cytomegalovirus promoter (SCMV) drives the expression of the HIV Gag and Pol Core proteins, where the Integrase is mutated and not functional. The Phosphoglucokinase (PGK) promoter drives the expression of Malaria antigen proteins, which can be chimeric (eg HA transmembrane-Malaria Ag) which if plural, are all separated by 2A or IRES sequences (not shown). The Post Transcriptional Regulatory Element (PRE) is optionally inserted distal to the Envelope ORF and poly A.

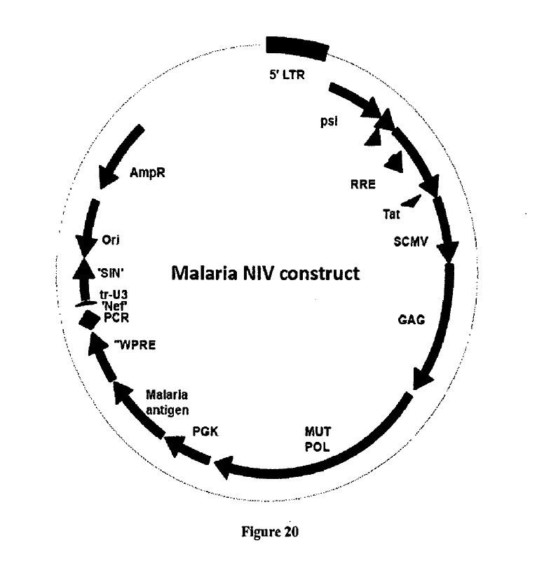

FIG. 21 shows a Malaria NIV vaccine construct that is comprised of a 5′LTR with a psi packaging sequence, RRE element and optionally some non-coding Tat sequence. The Cytomegalovirus promoter (SCMV) drives the expression of the HIV Gag and Pol Core proteins, where the Integrase is mutated and not functional. The Phosphoglucokinase (PGK) promoter drives the expression of Malaria antigen proteins, which can be chimeric (eg HA transmembrane-Malaria Ag) and a cytokine, which are all separated by 2A or IRES sequences (not shown). The Post Transcriptional Regulatory Element (PRE) is optionally inserted distal to the Envelope ORF and poly A.

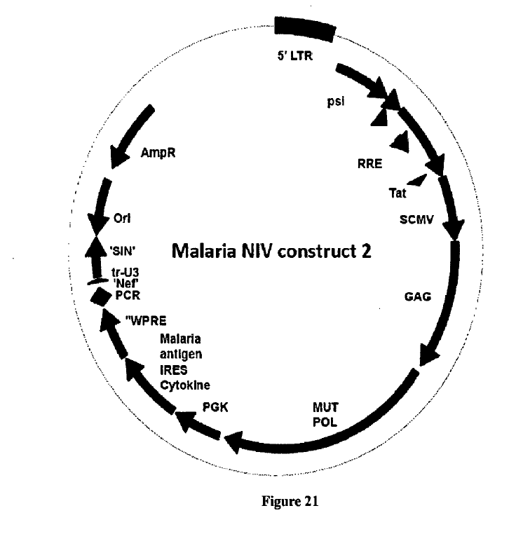

FIG. 22 shows a Bacterial NIV vaccine construct that is comprised of a 5′LTR with a psi packaging sequence, RRE element and optionally some non-coding Tat sequence. The Cytomegalovirus promoter (SCMV) drives the expression of the HIV Gag and Pol Core proteins, where the Integrase is mutated and not functional. The Phosphoglucokinase (PGK) promoter drives the expression of bacterial antigen proteins, which can be chimeric (eg VSV-G transmembrane-Bacterial Ag) which if plural, are all separated by 2A or IRES sequences (not shown). The Post Transcriptional Regulatory Element (PRE) is optionally inserted distal to the Envelope ORF and poly A.

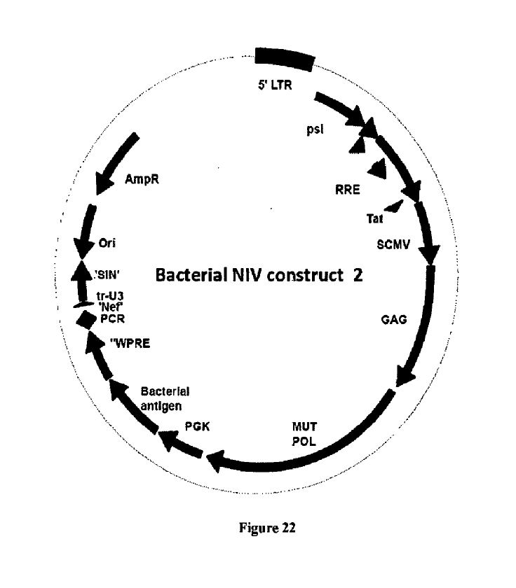

FIG. 23 shows a General NIV vaccine construct that is comprised of a 5′LTR with a psi packaging sequence, RRE element and optionally some non-coding Tat sequence. The Cytomegalovirus promoter (SCMV) drives the expression of the HIV Gag and Pol Core proteins, where the Integrase is mutated and not functional. The Phosphoglucokinase (PGK) promoter drives the expression of an antigen of interest proteins and a CTL epitope (of the target pathogen), which are all separated by 2A or IRES sequences (not shown). The Post Transcriptional Regulatory Element (PRE) is optionally inserted distal to the Envelope ORF and poly A.

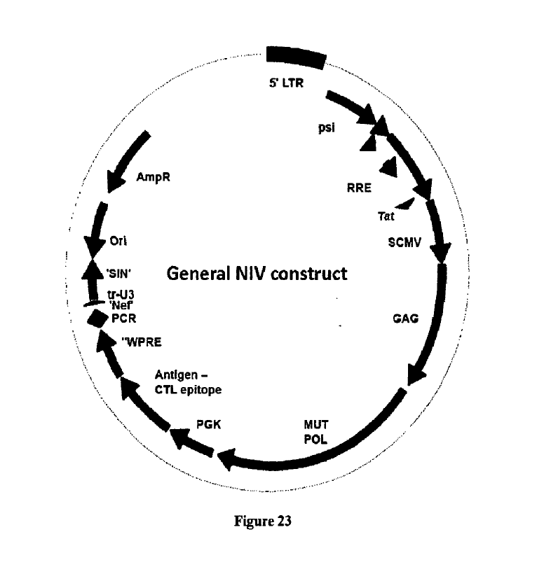

FIG. 24 shows a General NIV vaccine construct that is comprised of a 5′LTR with a psi packaging sequence, RRE element and optionally some non-coding Tat sequence. The Cytomegalovirus promoter (SCMV) drives the expression of the HIV Gag and Pol Core proteins, where the Integrase is mutated and not functional. The Phosphoglucokinase (PGK) promoter drives the expression of an antigen of interest proteins and a cytokine, which are all separated by 2A or IRES sequences (not shown). The Post Transcriptional Regulatory Element (PRE) is optionally inserted distal to the Envelope ORF and poly A.

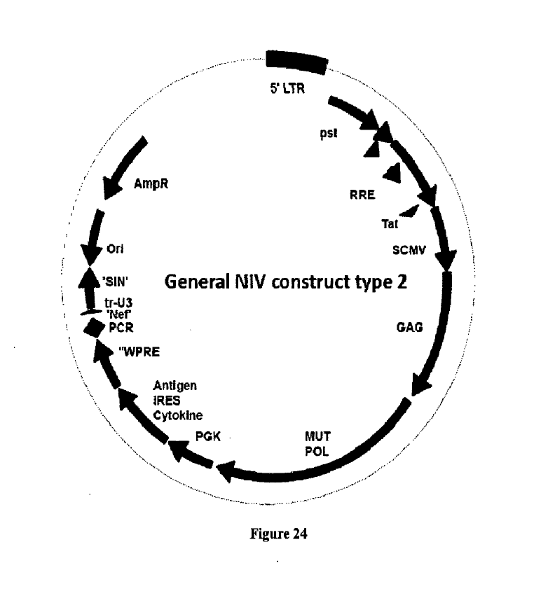

FIG. 25 shows a General NIV vaccine construct that is comprised of a 5′LTR with a psi packaging sequence, RRE element and optionally some non-coding Tat sequence. The Cytomegalovirus promoter (SCMV) drives the expression of the HIV Gag and Pol Core proteins, where the Integrase is mutated and not functional. The Phosphoglucokinase (PGK) promoter drives the expression of an antigen of interest proteins and a shRNAi that modulates the vaccines immunogenicity. The Post Transcriptional Regulatory Element (PRE) is optionally inserted distal to the Envelope ORF and poly A.

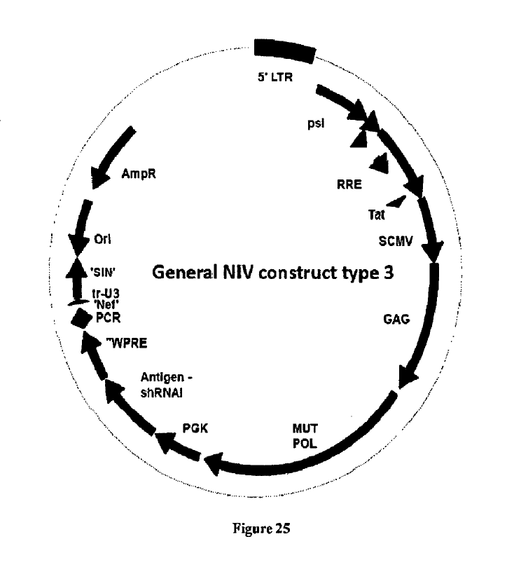

FIG. 26 shows a General NIV vaccine construct that is comprised of a 5′LTR with a psi packaging sequence, RRE element and optionally some non-coding Tat sequence. The Cytomegalovirus promoter (SCMV) drives the expression of the HIV Gag and Pol Core proteins, where the Integrase is mutated and not functional. The Phosphoglucokinase (PGK) promoter drives the expression of an antigen of interest proteins, a CTL epitope and a cytokine, which are all separated by 2A or IRES sequences (not shown). The Post Transcriptional Regulatory Element (PRE) is optionally inserted distal to the Envelope ORF and poly A.

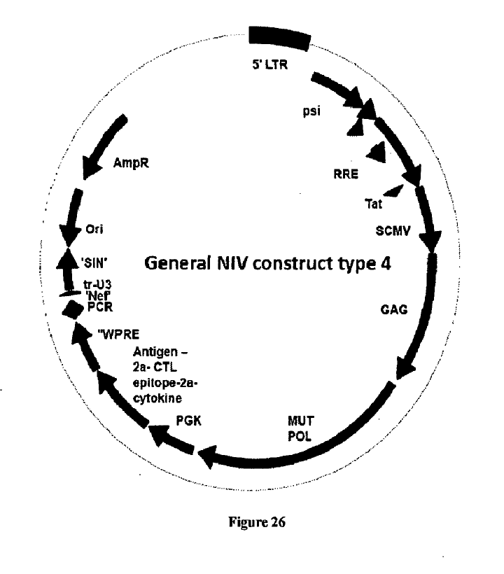

### DETAILED DESCRIPTION OF THE INVENTION

This invention relates to non-integrating, non-replicating retroviral vectors that cause an immune response in an animal host when administered to the host. The vectors transduce cells in the host, where they produce virus-like particles (VLPs), which stimulate an additional immune response in the host when they are released from the cells.

The retroviral vectors comprise long terminal repeats, a packaging sequence, and a heterologous promoter operably linked to one or more polynucleotide sequences that together encode the structural proteins of a virus. The virus is one to which it is desired to create an immunological response in a mammal. Thus, the retroviral vectors are a vaccine to this target virus. In one embodiment, the structural proteins are encoded by a retroviral gag gene. The vector is packaged by a helper construct that does not contain an integrase gene or contains an integrase gene that is not functional. Alternatively, the vector contains a retroviral pol gene that comprises a mutated integrase gene that does not encode a functional integrase protein. Such mutated integrase genes and constructs containing them can be prepared by techniques known in the art. In one embodiment, the vectors are self-inactivating (SIN) vectors, which have an inactivating deletion in the U3 region of the 3′ LTR. In one embodiment, the vectors are gammaretroviral vectors. In another embodiment, they are lentiviral vectors. In a particular embodiment, the lentiviral vectors are HIV vectors. As used herein, the term “HIV” includes all clades and/or strains of human immunodeficiency virus 1 (HIV-1) and human immunodeficiency virus 2 (HIV-2).

After the vector transduces a host cell, the polynucleotide sequences that encode the structural proteins of the virus are expressed and the structural proteins are produced. The vector comprises sufficient numbers and types of polynucleotide sequences for the structural proteins to self-assemble into VLPs. In one embodiment, the structural proteins comprise the viral capsid proteins, and the VLP is the viral capsid. In another embodiment, the structural proteins also include viral envelope proteins for those target viruses that have envelopes, and the VLP comprises the viral capsid and the viral envelope.

The viruses for which structural proteins are produced can be any virus for which the vectors of the invention can produce self-assembling structural proteins that form a VLP. These include lentiviruses, other retroviruses, influenza viruses, hepatitis viruses, filoviruses, and flaviviruses. More generally, these include viruses from the following families: Adenoviridae, Arenaviridae, Astroviridae, Baculoviridae, Bunyaviridae, Calciviridae, Coronaviridae, Filoviridae, Flaviridae, Hependnaviridae, Herpesviridae, Orthomyoviridae, Paramyxoviridae, Parvoviridae, Papovaviridae, Picornaviridae, Poxviridae, Reoviridae, Retroviridae, Rhabdoviridae, and Togaviridae. In particular embodiments, the viruses are selected from the group consisting of HIV-1, HIV-2, SIV, influenza A virus, influenza B virus, hepatitis A, B & C virus, Ebola virus, Marburg virus, West Nile virus and dengue fever virus. Thus, the retroviral vectors of the invention, after transducing cells of a host animal, produce VLPs that have the antigenic characteristics of the above-mentioned viruses as well as any other viruses for which VLPs can be produced in this manner. This permits their use as vaccines.

The capsid and envelope proteins encoded by the polynucleotide sequence in the vector can be from the same or different viruses. For example, the capsid and envelope proteins are derived from a single type of virus; the capsid proteins are from one type of virus and the envelope proteins are from another type of virus; or the capsid proteins are from one type of virus and the envelope proteins are from multiple types of viruses. The multiple types of envelope proteins can be derived from different strains of the same type of virus, or they can be derived from different strains of different virus types.

In one embodiment, the vectors contain a heterologous polynucleotide sequence that codes for a heterologous protein. That is, the protein is not part of the native retrovirus from which the vector is derived. The vectors may also contain two or more different heterologous sequences that code for different heterologous proteins. These proteins are part of the VLPs after the vector polynucleotide sequences are expressed in the host cells.

The heterologous protein can be any envelope protein that is capable of pseudotyping with a retroviral vector. A well-known example is the VSV-G protein. Other examples include Hepatitis C envelope proteins E1 and/or E2 and Dengue Virus E proteins, Baculovirus envelope protein, HIV envelope proteins, Amphotropic Retrovirus envelope proteins, Alphavirus envelope proteins, Flavivirus envelope proteins, and any other envelope protein from a virus. The viral envelope protein can be a chimeric protein, in that it contains the transmembrane domain of one protein and the extracellular domain of a second protein. Also, the invention is not restricted to the expression of a single proteins, but may include a plurality of proteins. For example for Influenza, both the Hemagglutinin and Neuraminidase envelope proteins are expressed in addition to the M1 protein to make the VLP. Any protein combination may be relevant, whether they are from the same virus or different viruses.

The heterologous polynucleotide can also code for an antigen, an immunomodulating protein, an RNAi, or a polypeptide that can induce cell death. It can code for any factor involved in a disease process in a mammal. These proteins can be made as single proteins or as chimeras.

The antigen can be any protein or part thereof. It can be derived from a virus, bacteria, parasite, or other pathogen. It can also be a tumor antigen, such as a cell membrane protein from a neoplastic cell.

The immunomodulating protein is any protein that is involved in immune system regulation or has an effect upon modulating the immune response. In one embodiment, it is a cytokine, such as an interleukin, an interferon, or a tumor necrosis factor. In one aspect, the cytokine is IL-2, IL-12, GM-CSF, or G-CSF.

A polypeptide that can induce cell death is a polypeptide in a cell that directly or indirectly kills the cell when it is exposed to certain chemicals, such as certain drugs. Examples are known to those skilled in the art and include thymidine kinase (TK), deoxycytidine kinase (dCK), and the modified mammalian and human thymidylate kinase (TMPK) disclosed in U.S. patent application Ser. No. 11/559,757, filed on Nov. 14, 2006 and published on Mar. 19, 2009 as US 2009/0074733 A1, which is incorporated herein by reference in its entirety.

The NIV can also include an RNAi sequence (any gene inhibitory sequence also including an antisense, ribozyme, etc.) that is generally in the form of a shRNA (expressed within the cell upon the NIV expressing its genome in the cell). These sequences are expressed to either inhibit wild-type virus infection of the cell that produces VLPs, to enhance the immune response to the vaccine, to enhance production of the VLP proteins, to change the glycosylation of the VLP (eg deglycosylate the surface proteins by targeting proteins involved in their glycosylation) or to increase or decrease the longevitiy of the cell via targeting of apoptotic or cell survival gene pathways.

In one embodiment, the polynucleotide sequences are codon optimized. Codon optimization is well known in the art. It involves the modification of codon usage so that higher levels of protein are produced. Also, codon optimization may be used to degenerate the codon usage for a particular purpose. One example of this is to degenerate the codon usage with respect to the wild type virus so that there is no opportunity for the NIV to recombine with a wild type virus that infects the same cell producing the VLP.

As mentioned above, the retroviral vectors include lentiviral vectors. These vectors can be constructed from any lentivirus by techniques known to those skilled in the art, given the teachings contained herein. In one embodiment, the lentiviral vector is an HIV vector, constructed from HIV-1 or HIV-2 or a combination thereof. In another embodiment, it is an SIV vector, constructed from SIV. All of these vectors have the characteristics of the retroviral vectors discussed above. Of course, they are derived from the particular lentivirus in question instead of other retroviruses.

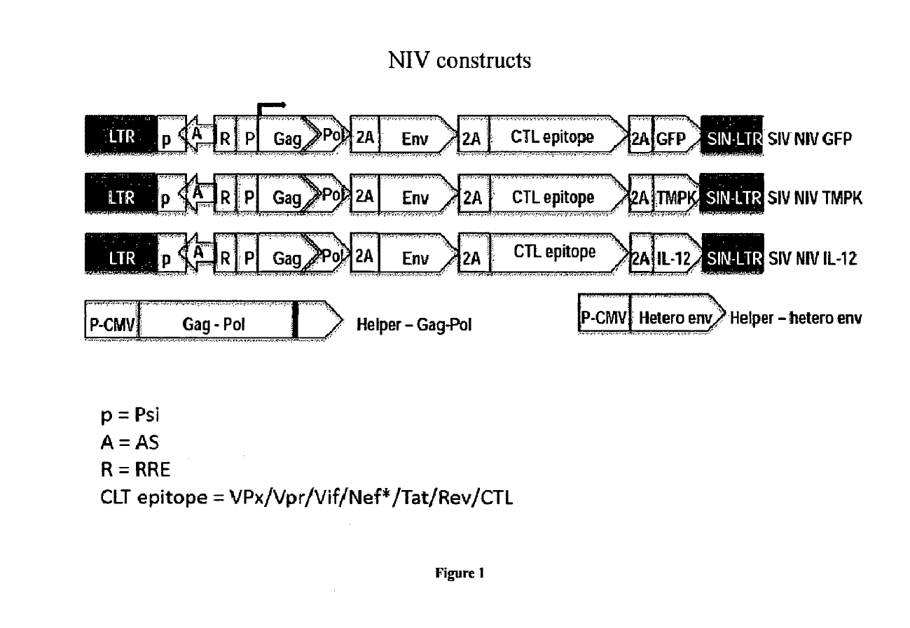

The skilled artisan will recognize that there will be certain differences in the lentiviral constructs, particularly those involving HIV or SIV, as compared to other retroviral vectors, such as gammaretroviral vectors. For example, for the production of an AIDS vaccine, the vector will preferably include an HIV gag gene for expression of HIV structural proteins in cells transduced by the vector. The complete gag gene will express the HIV capsid, nucleocapsid, and matrix proteins. In addition, the HIV vector will preferably include an HIV env gene so that the VLPs contain the envelope proteins. The vectors can also be pseudotyped, for example with VSV-G or Dengue E protein. Then they can be used to get transduction of non-CD4 cells as the HIV protein only infects CD4 T cells Preferably, it will also be a SIN vector. The vector may also include a nucleotide sequence that encodes a polypeptide that consolidates the major cytotoxic T-lymphocyte (CTL) and humoral B cell epitopes of different HIV clades or strains. In one embodiment, this is the Vpx/Vpr/Vif/Nef*/Tat/Rev/CTL polypeptide sequence shown in FIG. 1.

In one embodiment, the HIV vectors preferably contain a pol polynucleotide sequence from which the integrase gene has been deleted or in which the integrase gene has been mutated by deletion or modification of some of the wild type nucleotides. Thus, it cannot encode a functional protein. In one aspect of this embodiment, the modified integrase gene encodes an integrase protein with mutations at least one of amino acids D64, D116, and E152. In one particular aspect, the mutation is the D64 mutation.

The HIV vectors can also include an anti-Pol antisense sequence. In one aspect, the sequence is about 800 nucleotides long.

Without limiting the nature of the invention, the NIV can express a number of combinations of proteins to have their immunogenic effects. They can express either core and other proteins from a retrovirus (including Lentivirus and other viruses of the Retroviridae family) or core proteins from the virus which is the target for the vaccine. In the case of using retrovirus core and other proteins, the integrase of the retrovirus should preferably be inactivated. However, it is also possible that integration of the vector could be prevented by other means, including disruption of the att (attachment) sites of the LTR. Alternatively and preferably, the core protein of the target virus is used (eg for Influenza it is the M1 and possibly the M2 proteins; for Dengue Fever Virus is the pM protein and so forth). If the NIV encodes the core of the target virus, the NIV should be produced using a helper construct to package the NIV. This helper construct should at least contain retroviral Gag and Pol enzymes needed for reverse transcription and integration and has to be integrase defective. The NIV may optionally be pseudotyped with a heterologous envelope protein to facilitate transduction of the particle so that it can then express VLP native to the target virus.

The invention also includes pharmaceutical compositions. The compositions comprise one or more of the vectors of the invention in a pharmaceutically acceptable carrier. Such carriers are known to those skilled in the art. An example is an isotonic buffer that comprises lactose, sucrose or trehalose. The compositions may also include one or more adjuvants. Such carriers are also known to those skilled in the art. Examples include one or more of the following: alum, lipid, water, buffer, peptide, polynucleotide, polymer and/or an oil.

The vectors of the invention are constructed by techniques known to those skilled in the art, given the teachings contained herein. Techniques for the production of retroviral vectors are disclosed in U.S. Pat. Nos. 4,405,712, 4,650,746, 4,861,719, 5,672,510, 5,686,279, and 6,051,427, the disclosures of which are incorporated herein by reference in their entireties. Techniques for the production of lentiviral vectors are disclosed in U.S. patent application Ser. No. 11/884,639, published as US 2008/0254008 A1, and in U.S. Pat. Nos. 5,994,136, 6,013,516, 6,165,782, 6,294,165 B1, 6,428,953 B1, 6,797,512 B1, 6,863,884 B2, 6,924,144 B2, 7,083,981 B2, and 7,250,299 B1, the disclosures of which are incorporated herein by reference in their entireties.

The enveloped vector particle may be pseudotyped with an engineered or native viral envelope protein from another viral species, including non-retroviruses and non-lentiviruses, which alters the host range and infectivity of the native retrovirus or lentivirus. The envelope polypeptide is displayed on the viral surface and is involved in the recognition and infection of host cells by the virus particle. The host range and specificity can be changed by modifying or substituting the envelope polypeptide, e.g., with an envelope expressed by a different (heterologous) viral species or which has otherwise been modified. See, e.g., Yee et al., Proc. Natl. Acad. Sci. USA 91: 9564-9568, 1994, which is incorporated herein by reference in its entirety. Vesicular stomatitis virus (VSV) protein G (VSV-G) has been used extensively because of its broad species and tissue tropism and its ability to confer physical stability and high infectivity to vector particles. See, e.g., Yee et al, Methods Cell Biol., (1994) 43:99-112, which is incorporated herein by reference in its entirety. Examples of an envelope polypeptide that can be utilized include, e.g., Dengue fever virus, HIV gp120 (including native and modified forms), Moloney murine leukemia virus (MoMuLV or MMLV), Harvey murine sarcoma virus (HaMuSV or HSV), murine mammary tumor virus (MuMTV or MMTV), gibbon ape leukemia virus (GaLV or GALV), Rous sarcoma virus (RSV), hepatitis viruses, influenza viruses, Moloka, Rabies, filovirus (e.g., Ebola and Marburg, such as GP1/GP2 envelope, including NP.sub.—066246 and Q05320), amphotropic, alphavirus, etc. Other examples, include, e.g., envelope proteins from Togaviridae, Rhabdoviridae, Retroviridae, Poxyiridae, Paramyxoviridae, and other enveloped virus families. Other examples of envelopes from viruses are listed in the following database located on the worldwide web at ncbi.nlm.nih.gov/genomes/VIRUSES/viruses.html. Use of certain envelope proteins permit the retroviral vectors to transduce cells other than CD4 T cells, such as dendritic and other antigen-presenting cells.

The present invention includes plasmids, helper constructs, and producer cells used to construct and produce the vectors of the invention. The plasmid comprises retroviral long terminal repeat sequences, a retroviral packaging sequence, and a heterologous promoter operably linked to one or more polynucleotide sequences that together encode the structural proteins of a virus. In one embodiment, the retroviral sequences are lentiviral sequences. In one aspect of this embodiment, the lentiviral sequences are HIV sequences.

The packaging cell comprises the plasmid of the invention and a helper construct that does not contain an integrase gene or contains an integrase gene that is not functional. In one embodiment, the cell is a mammalian cell. In one aspect, it is a simian cell. In another aspect, it is a human cell. Examples include known cell lines like 293 cells, PER.C6 cells, stem cell lines, embryonic or neonatal cell lines, cells derived from the umbilical cord or any human or mammalian cell line. The packaging cells are made by transfecting a human or mammalian cell or cell line with the plasmid of the invention and the appropriate helper constructs if required.

The producer cell comprises the plasmid of the invention and a helper construct that does not contain an integrase gene or contains an integrase gene that is not functional. In one embodiment, the cell is a human or mammalian cell. In one aspect, it is a simian cell. In another aspect, it is a human cell. Examples of cells include those mentioned in the preceding paragraph. The producer cells are made by transfecting a mammalian cell or cell line with the plasmid of the invention and the appropriate helper constructs. These cells are cultured and produce the vectors continuously or in batches. If VSV-G is used, the cells can only be induced to produce VSV-G decorated particles for a limited time as the protein is toxic to cells when expressed to high levels. The vectors are recovered from the supernatant by known techniques. Producer cells lines constitutively producing enveloped vaccine vector particles can be produced using envelope proteins other than the native VSV-G protein.

The vectors and pharmaceutical compositions of the invention are used as vaccines. Thus, the invention includes a method of causing an immune response in a mammal by delivering the vectors or the pharmaceutical compositions to the mammal in an amount sufficient to cause an immune response in the mammal. The vectors and compositions are delivered by means known in the vaccine art. For example, they may be delivered subcutaneously or intramuscularly, such as by injection. After the vectors transduce cells in the mammal, the cells produce and release VLPs comprising the structural proteins of the virus, causing a further immune response in the mammal. In one embodiment, the mammal is a laboratory animal. For example, it can be a rodent, such as a mouse, rat, or guinea pig, a dog or cat, or a non-human primate. In another embodiment, the mammal is a human.

The VLPs comprise the structural proteins of a virus. The virus is any virus for which the vectors of the invention can produce self-assembling structural proteins that form a VLP. These include lentiviruses, other retroviruses, influenza viruses, hepatitis viruses, filoviruses, flaviviruses or any of the virus derived from families described above in this application. In particular embodiments, the viruses are selected from the group consisting of HIV-1, SIV, Seasonal and Pandemic Influenza, including Influenza A virus and Influenza B virus strains, Hepatitis A, B or C virus, Arbovirus infections including West Nile Virus, Ebola virus, Cytomegalovirus, Respiratory Syncitial virus, Rabies virus, Corona virus infections, including SARS, Human Papilloma virus, Rotaviruses, Herpes Simples Virus, Marburg virus, and dengue fever virus. The structural proteins comprise the core of the virus. They can also include the envelope of the virus.

The core and envelope proteins can be from the same or different viruses. For example, the capsid and envelope proteins are derived from a single type of virus; the capsid proteins are from one type of virus and the envelope proteins are from another type of virus; or the capsid proteins are from one type of virus and the envelope proteins are from multiple types of viruses. The multiple types of envelope proteins can be derived from different strains of the same type of virus, or they can be derived from different strains of different virus types. In the case of HIV or SIV, the structural proteins are one or more of the capsid proteins and/or the nucleocapsid proteins and/or the matrix proteins.

The envelope protein is any protein that is capable of pseudotyping with a retroviral vector and becoming part of the VLP. Examples include the VSV-G protein and the Hepatitis C envelope proteins E1 and E2, Influenza virus HA and NA proteins, Dengue Fever Envelope proteins, Ross River Virus Envelope proteins, Semliki Forest Virus Envelope proteins, Sindbis Virus Envelope proteins, HIV or SIV envelope proteins, Mokola virus envelope proteins, and retroviral amphotropic envelope proteins. These envelope proteins can be derived from any virus, or could be synthesized de novo as chimeric or novel envelope proteins.

As mentioned above, the vectors can include a heterologous polynucleotide sequence that encodes an antigen, an immunomodulating protein, an RNAi, or a polypeptide that can induce cell death. In such case, the VLPs will include the antigen, immunomodulating protein, RNAi sequence, or a polypeptide that can induce cell death.

The antigen can be any protein or part thereof. It can be derived from a virus, bacteria, parasite, or other pathogen. It can also be a tumor antigen, such as a cell membrane protein from a neoplastic cell. It can also be a tumor antigen that is not on the cell membrane. In such cases, such tumor antigens are either incorporated with transmembrane domains, so that they are expressed on the surface of the particles, or they are singly expressed within the cell without linkage to any other protein. The tumor antigens can also be linked to other protein or peptide sequences that increase the immunogenicity of the tumor antigen. Such sequences are known in the art and they generally stimulate native immunity through TLR pathways.

The immunomodulating protein is any protein that upregulates, downregulates, or modulates the host's immune response towards the target antigen of the NIV vaccine. In one embodiment, it is a cytokine, such as an interleukin, an interferon, or a tumor necrosis factor. In one aspect, the cytokine is IL-2, IL-12, GM-CSF, or G-CSF. Other cytokine examples that modulate the immune response that could be incorporated are found at www.ncbi.nlm.nih.gov. Immunomodulating protein are not only restricted to cytokines. They can be other proteins such as ligands or protein fragments that act as ligands. They can also be comprised of antibodies that target ligand binding sites on target proteins on cells. One example of antibodies and ligands are CTLA-4 antibodies and the CD-40L protein. Other examples are found in the art and some can be found at www.ncbi.nlm.nih.gov.

A polypeptide that can induce cell death is a polypeptide in a cell that directly or indirectly kills the cell when it is exposed to certain chemicals, such as certain drugs. As mentioned above, examples include thymidine kinase (TK), deoxycytidine kinase (dCK), and the modified mammalian and human thymidylate kinase (TMPK). Other polypeptides that can induce cell death can be found at www.ncbi.nlm.nih.gov.

In one preferred embodiment, the vector of the invention is an HIV SIN vector comprising an HIV LTR, an HIV packaging sequence, and a heterologous promoter, such the CMV promoter, the EF1-alpha promoter, the MND promoter, the PGK promoter, operably linked to an HIV gag sequence and an HIV pol sequence. The pol sequence contains an integrase sequence that does not encode a functional integrase protein (integrase-ve). Alternatively, the integrase sequence could have been deleted. In one aspect, the heterologous promoter is also operably linked to an HIV env sequence that encodes the gp120/41 envelope proteins. In another aspect, this vector is pseudotyped with a second type of helper construct that expresses an envelope protein, examples being the VSV-G envelope proteins, Mokola virus envelope protein, amphotropic retrovirus envelope protein, or Dengue Fever virus envelope proteins, to facilitate transducion into a larger number of cells, such as antigen presenting cells. The vector is produced by packaging or producer cells and contain the following constructs: the first construct is the vector construct that expresses the GagPol (integrase-negative) structural and enzymatic (protease and reverse transcriptase) proteins of HIV and using a second promoter on the same vector construct, the HIV gp120/41 (of any strain or from multiple strains or clades); the second construct (second type of helper) expresses one of the heterologous env proteins described above from a heterologous promoter and preferably is expressed only to levels sufficient for pseudotyping and facilitating increased transduction of the NIV into cells, preferably antigen presenting cells (eg dendritic cells).

In another preferred embodiment, the vector of the invention is an HIV SIN vector comprising an HIV LTR, an HIV packaging sequence, and a heterologous promoter, such as such the CMV promoter, the EF1-alpha promoter, the MND promoter, or the PGK promoter, operably linked to a polynucleotide sequence encoding Hepatitis C virus structural and envelope proteins. In one aspect, a second helper construct is used in addition to the first helper construct that expresses the GagPol (Integrase-negative) proteins. This second helper construct expresses an envelope protein of a virus that can pseudotype with the NIV such as the VSV-G envelope proteins, Mokola virus envelope protein, amphotropic retrovirus envelope protein, or Dengue Fever virus envelope proteins to facilitate transduction into a larger number of cells, such as antigen presenting cells. These pseudotyping envelope proteins should not be encoded in the vector as they would distract the immune response if expressed during VLP production, after transduction of cells with the NIV in the body.

In one aspect, the second helper vector expressing pseudotype envelope proteins is not used, particularly if the envelope of the target virus is able to pseudotype with the NIV and allow NIV transduction of antigen presenting cells. This is true of Dengue Fever virus envelope proteins. Therefore, a second helper expressing pseudotyping envelope proteins would not be required for a Dengue Fever Virus NIV vaccine. It is known that Dengue Fever virus envelope proteins are able pseudotype with HIV based Lentiviral vectors, which are able to efficiently transduce antigen presenting cells like dendritic cells. The same is true for many other viruses that would be vaccine targets; however, it is not true in every case, and in those cases it would be preferable to use the second helper construct expressing a pseudotyping envelope protein to facilitate NIV transduction of antigen presenting cells right after administration of the vaccine. It should be noted that the proteins expressed from all helper constructs (the first type—structural, and the second type—envelope) are not encoded in the vector, and therefore would not be antigenic beyond initial introduction of the vaccine after injection. Persistent immune responses are generated from the VLPs that are produced from the NIVs in the body, and since they are wholly (in some cases almost wholly) native, the immune response is trained to be highly specific for the targeted virus.

The following example illustrates certain aspects of the invention and should not be construed as limiting the scope thereof.

### EXAMPLE

This example shows the construction of a non-integrating lentiviral vector vaccine. An NIV derived from an HIV should be especially promising as AIDS vaccine for the reasons stated in the Background section above. As a means of demonstrating efficacy and proof-of-principle, an SIV NIV will be developed first, and later transitioned into the HIV version.

The vaccine will be produced by the use of plasmids introduced into producer cells: the NIV plasmid, containing all relevant antigens, and two helper constructs, the first expressing Gag, mutated Pol and VSV-G (for priming), and the second expressing Gag and mutated Pol only (for boosting). See FIG. 1. The resultant replication-defective NIV particle will be used as a vaccine to transduce cells and express HIV proteins and produce NIV VLPs to activate the immune system. This feature would mimic HIV infection, without the sequelae following live virus exposure.

A prototype NIV would contain the following safety features to make it safe for use in the general population. These vectors will be engineered to eliminate any possibility of reversion or adverse recombination with wild-type HIV:
(a) The NIV is non-replicating and can only transduce cells and express HIV proteins and HIV-like particles (VLP), but cannot replicate beyond this single round because it is deleted in essential proteins and cis-acting elements that are required for replication (as described more specifically below).
(b) The NIV would be produced using a mutant-integrase helper. Therefore, while the vector genome will enter and persist in non-dividing cells, it is non-integrating. A combination of three mutations within the integrase gene would be used. Alternatively, mutant vector attachment sites could be used solely or in combination with the mutant integrase.
(c) Expression of HIV gag, env and other HIV proteins in a codon optimized/degenerate manner to eliminate their cis-acting elements and increase protein expression.
(d) The vector would additionally contain an anti-Pol antisense sequence to prevent recombination with the helper and inhibit wt-HIV replication in cells that would become co-infected with wt-HIV. The antisense would be about 800 bases in length and targeted to the Pol gene, a highly conserved region of HIV.
(e) The vector would preferably contain codon degerated mutant Pol sequences, or not contain any Pol sequences so that, if recombination should occur with wt-HIV, the result would be a non-functional virus; the mutant Pol (mutant integrase) gene functions would be expressed only from the helper during production.
(f) The NIV 3′ LTR would be deleted in the promoter and enhancer regions so that the result is a self-inactivating (SIN) vector; therefore no native LTR would be present in vaccinated individuals' cells. (The 3′LTR is double-copied during reverse transcription, resulting in two copies of the enhancer/promoter deleted LTR.)
(g) Presence of a human safety gene (e.g. human mutant TMPK, a type of human TK gene) in the vector that would allow transduced cells to be eliminated, if required, by oral administration of AZT.
   The antigenic features of a basic and optimized NIV are as follows:
(a) The NIV, being HIV, would not induce a non-HIV immune response during repeated injection, as the NIV is not a heterologous vector to the virus targeted for vaccination.
(b) Use of a highly active promoter (e.g. EF-1alpha, CMV, MND) to express codon-optimized HIV genes (Gag, Env, Rev, Tat, Vpu, Vpr, Nef, but preferably not Pol), or variants of these proteins, would result in high levels of HIV protein expression.
(c) By expressing all these proteins, the NIV would also produce HIV VLPs from transduced cells, potentially increasing the immunogenicity of the vaccine.
(d) The NIV would be pseudotyped with VSV-G for priming, so that dendritic and other cell types are transduced to persistently express HIV proteins.
(e) The NIV used for boosting would use either an alternative envelope pseudotype, or preferably no pseudotype (only native gp120/41 envelope) in order to target cells that would normally be targeted by wt-HIV.
(f) The NIV could optionally express a polypeptide that consolidates major CTL epitopes of HIV strains (or clades).
(g) The NIV could optionally express genes (e.g. IL-12 or IL-15) or suppressive factors (e.g. miRNA/RNAi) that could enhance/modulate the immunogenicity or persistence of the vaccine.
(h) A mixture of HIV vaccine vector strains could be developed; antigenic competition would need to be evaluated

### Experimental

An NIV will be developed and shown to express in vitro. These NIVs will then be used for immunogenicity testing in non-human primates. The SIV239Mac model system will be used.

(1) Design SIV NIV that expresses SIV proteins. SIV 239Mac are used as the backbone sequence for the vector. The entire NIV can be synthesized and cloned into a stable plasmid backbone. An EF1alpha or CMV promoter expresses codon-optimized SIV Gag, Env, Tat, Rev, Vif, Vpr, Vpx and Nef. NIVs without genes encoding the accessory proteins will also be developed. An antisense targeted to the integrase gene can also be cloned into the NIV. Helper constructs can also be similarly synthesized, expressing the Pol genes and the mutant integrase gene, optionally with multiple mutations. Helper constructs will express the Gag-mutant-Pol (integrase negative) genes and will either express or not express the VSV-G or Dengue E protein.

(2) Synthesize SIV NIV that expresses SIV proteins. The vector and helper plasmid DNA constructs are synthesized using methods known in the art.

(3) Manufacture SIV NIVs. Vector and helper constructs can be transfected into 293 cells, and the NIV vector particles concentrated and purified. Given the similarity of SIV and HIV particles, the production, concentration, and purification methods should be very similar.

(4) Test SIV NIVs for transduction and expression of HIV proteins in CEM cells. CEM cells can be transduced with SIV NIV particles to demonstrate transduction and expression of HIV proteins. Transduction is monitored by copy number, using a unique sequence that will be included in the NIV. A quantitative PCR assay is used to measure for transduction efficiency of vectors with this sequence. Protein expression is ascertained by western blot and FACS analysis.

(5) Test SIV NIVs for transduction and expression of HIV proteins in macaque cells. The NIV is tested in primary Macaque PBLs for transduction and protein expression by the methods described above.

(6) Demonstrate the Safety of NIVs. The safety of SIV NIVs can be demonstrated by several methods. First, the supernatants of CEM cells transduced with SIV NIV is filtered and then passaged onto naive CEM cells to test for the presence of replication competent virus. Similarly, supernatants from primary cells transduced with NIV is then tested for replication competent virus on CEM cells. To analyze the frequency of integration into the genome, genomic DNA will be isolated from the cells and quantitative PCR will be performed. If integration is shown to occur, the integration sites will be mapped by inverse PCR.

(7) Demonstrate the anti-SIV effects of SIV NIVs. The SIV NIVs will be engineered to express an anti-Pol antisense sequence targeted to wt-SIV. Other antisense sequences can be designed and tested. The anti-SIV effects of NIV will be determined by challenging transduced CEM cells with wt-SIV, and the level of inhibition of wt-SIV replication will be measured by p27 ELISA assay.

(8) Process development and the manufacture of SIV NIVs under GLP conditions. Existing processes for the manufacture and production of GMP HIV-based Lentiviral vectors can be adapted for the production of SIN NIV under GLP conditions. A combination of tangential flow filtration and ionic exchange chromatography has been successfully used for the concentration and purification of HIV-based lentiviral vectors. After manufacture, the GLP NIV material will be tested for several tests prior to release for the animal studies.

(9) Demonstrate the immunogenicity of SIV NIVs in non-human primates. Animals would be injected with the SIV NIV GLP material and tested for immunogenicity. Indian-origin rhesus macaques will be injected subcutaneously with 10⁷, 10⁸ or 10⁹ infectious units, in groups of 3 each. Plasma viremia will be quantified by RT-PCR; animals will also be assessed for the presence of replication-competent virus as described below. Immune responses will be evaluated by: 1) Interferon-gamma ELISPOT assays of PBMC using overlapping peptide pools corresponding to all SIV proteins; 2) intracellular cytokine staining assays using PBMC stimulated with Gag or Env peptide pools, evaluating 4 effector functions (secretion of IFN-gamma, TNF-alpha, and IL-2, and upregulation of CD107a) in CD4+ and CD8+ T cells); and 3) analysis of SIV-specific antibodies using gp140 ELISAs and neutralization of SIVmac251 and SIVmac239. Assuming immunogenicity is observed, animals will be challenged with a single, high intrarectal dose of SIVmac239 at 6 months after initial vaccination, and followed for plasma viremia, preservation of total and central memory CD4+ T cells responses, and SIV-specific humoral and cellular immune responses as described above.

(10) Demonstrate the safety of SIV NIVs in non-human primates. The biodistribution of SIV NIVs will be determined by taking biopsies from various tissues and assaying for the presence of SIV NIV DNA by PCR. To test for a putative replication competent Lentivirus (RCL), PBLs from non-human primates vaccinated with SIV NIVs would be isolated and co-cultured with CEM cells to determine for the presence of a RCL. The co-cultured cells will be assayed for the presence of an SIV by p27 assay and/or qPCR with appropriate positive and negative controls. Genomic DNA will also be isolated to determine the frequency of SIV NIV integration. If integration has occurred, the site of integration will by mapped by inverse PCR.

### SUMMARY

SIVΔNef is still the most potent candidate HIV vaccine ever developed. Although correlates for immunity are not precisely known, persistent expression of HIV proteins appears to be important. Also, it is known that expression of HIV proteins from heterologous vectors, such as adenoviral vectors, can be problematic and leads to a significant anti-vector immune response. This example illustrates a non-replicative HIV vector that does not integrate, but can express HIV antigens (and VLPs) at high levels from transduced cells. NIVs should have advantages over heterologous vectors expressing HIV proteins, since there would be no non-HIV proteins expressed to distract and bifurcate the immune system from generating a HIV specific immune response. Repeated vaccination with a NIV may produce effects similar to SIVΔNef, but without the safety issues observed with that attenuated virus.

A similar experimental approach can be adapted for the development of vaccines for other diseases. The constructs would contain formats that have been described in this application. These constructs are synthesized or cloned and then manufactured using the procedures described above. The vectors are then tested in animal models for safety and immunogenicity. For vaccines that are targeted to infections other than HIV, the NIV can express core and other viral proteins that are nascent to the pathogen causing virus of interest. The VLPs resulting from this NIV would not distract the immune response as the proteins are wholly from the pathogenic virus of interest.

### REFERENCE

Braun S E, Lu X V, Wong F E, Connole M, Qiu G, Chen Z, Slepushkina T, Slepushkin V, Humeau L M, Dropulic B, Johnson R P, Potent inhibition of simian immunodeficiency virus (SIV) replication by an SIV-based lentiviral vector expressing antisense Env, Hum. Gene Ther. 2007 Jul. 18(7):653-64.

Although this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

### REFERENCES

All publications, including issued patents and published patent applications, and all database entries identified by url addresses or accession numbers are incorporated herein by reference in their entirety.

1. A non-integrating, non-replicating lentiviral vector comprising a long terminal repeat, a packaging sequence, and a heterologous promoter operably linked to one or more polynucleotide sequences that together encode the structural proteins of a virus.

2-4. (canceled)

5. The vector of claim 1 wherein the structural proteins self-assemble into a virus-like particle (VLP) when the polynucleotide sequences are expressed in a cell transduced by the vector.

6. The vector of claim 1 wherein the vector comprises a self-inactivating (SIN) vector.

7. The vector of claim 1 wherein the virus is selected from the group consisting of lentivirus, influenza virus, hepatitis virus, alphavirus, filovirus, and flavivirus.

8. (canceled)

9. The vector of claim 1 wherein the structural proteins are the capsid of the virus.

10. The vector of claim 1 wherein the structural proteins further include the envelope of the virus.

11-15. (canceled)

16. The vector of claim 1 further comprising a heterologous polynucleotide sequence that encodes an immunostimulating protein.

17-18. (canceled)

19. The vector of claim 1 comprising a heterologous polynucleotide sequence that encodes a bacterial, viral, or tumor antigen.

20-38. (canceled)

39. The vector of claim 1 wherein the structural proteins are selected from the group consisting of the HIV gag protein, the influenza matrix protein, and the hepatitis core protein.

40-100. (canceled)

101. A method of causing an immune response in a mammal comprising delivering the lentiviral vector of claim 1 to the mammal in an amount sufficient to cause an immune response in the mammal wherein the lentiviral vector transduces cells in the mammal and the transduced cells produce and release a sufficient amount of VLPs comprising the structural proteins of the virus to cause a further immune response in the mammal.

102. The method of claim 101 wherein the vector is delivered subcutaneously or intramuscularly.

103-104. (canceled)

105. The method of claim 101 wherein the mammal is a human.

106-133. (canceled)

134. A VLP comprising the structural proteins of a virus.

135-144. (canceled)

145. The VLP of claim 134 further comprising a heterologous polypeptide comprising a bacterial, viral, or tumor antigen.

146-154. (canceled)

155. A VLP produced by a process comprising the step of infecting a mammalian cell in vivo with the vector of claim 1.

156. The VLP of claim 155 wherein the mammalian cell is a human cell.

157-161. (canceled)

162. The vector of claim 19 wherein the heterologous env gene is selected from the group consisting of a VSV-G env gene, influenza A virus env gene, influenza B virus env gene, hepatitis C virus env gene, Ebola virus env gene, Marburg virus env gene, and dengue fever virus env gene.

163-221. (canceled)
